(19) United States
(12) Patent Application Publication
(10) Pub. No.: US 2005/0084874 A1
(43) Pub. Date: Apr. 21, 2005

(54) Microfiltration and/or ultrafiltration process for recovery of target molecules from polydisperse liquids

(76) Inventors: Georges Belfort, Slingerlands, NY (US); Gautam Lal Baruah, Troy, NY (US)

Correspondence Address: Michael L. Goldman;Nixon Peabody LLP, Clinton Square, P.O. Box 31051, Rochester, NY, 14603-1051, US

(21) Appl. No.: 10/812,792

(22) Filed: Mar. 30, 2004

Related U.S. Application Data
(60) Provisional application No. 60/464,497, filed on Apr. 22, 2003.
(60) Provisional application No. 60/527,919, filed on Dec. 8, 2003.

(51) Int. Cl. C12Q 1/68; G01N 33/53; C07H 21/04

(52) U.S. Cl. 435/6; 435/7.1; 530/412; 536/25.4

(57) ABSTRACT

The present invention is directed to an improved microfiltration process, an improved ultrafiltration process, and an improved combination microfiltration processs/ultrafiltration process, all for recovering target molecules from a polydisperse liquid. These processes are particularly useful in recovering proteins from transgenic milk.

### FIELD OF THE INVENTION

This application claims the benefit of U.S. Provisional Patent Application Ser. Nos. 60/464,497, filed Apr. 22, 2003 and 60/527,919, filed Dec. 8, 2003.

The present invention relates to a microfiltration and/or ultrafiltration process for recovery of target molecules from polydisperse liquids.

### BACKGROUND OF THE INVENTION

The transgenic process is an economically attractive method of producing large amounts of human therapeutic proteins (John et al., “Expression of an Engineered Form of Recombinant Procollagen in Mouse Milk,” Nature Biotech. 17:385-389 (1999); Kreeger, “Transgenic Mammals Likely to Transform Drug Making,” The Scientist, 11(15):11 (1997); Mckee et al., “Production of Biologically Active Salmon Calcitonin in the Milk of Transgenic Rabbits,” Nature Biotech. 16:647-651 (1998); Pollock et al., “Transgenic Milk as a Method for the Production of Recombinant Antibodies,” J. Immunol. Methods 231:147-57 (1998); and Prunkard et al., “High-Level Expression of Recombinant Human Fibrinogen in the Milk of Transgenic Mice,” Nature Biotech. 14:867-71 (1996)). This involves the creation of genetically altered animals and plants such that the desired heterologous protein is recoverable in their milk, eggs, fruit, etc. A DNA construct that encodes the target human protein, is inserted into a goat cell line by transfection. The transfected transgenic cell is then fused with an enucleated oocyte by electrofusion. After 24-48 hours in culture, the embryo is transferred to a surrogate mother. The putative transgenic animals are identified by screening the offspring for the transgene. After the selected females mature, they are bred and the milk produced after gestation is tested for protein expression.

Transgenic versions of therapeutic proteins, implicated in chronic diseases, like human monoclonal antibodies, tissue plasminogen activator, antithrombin III, and human lactoferrin are in various stages of FDA approval (John et al., “Expression of an Engineered Form of Recombinant Procollagen in Mouse Milk,” Nature Biotech. 17:385-89 (1999); Kreeger, “Transgenic Mammals Likely to Transform Drug Making,” The Scientist, 11 (15):11 (1997); Mckee et al., “Production of Biologically Active Salmon Calcitonin in the Milk of Transgenic Rabbits,” Nature Biotech. 16:647-51 (1998); Pollock et al., “Transgenic Milk as a Method for the Production of Recombinant Antibodies,” J. Immunol. Methods 231:147-157 (1998); and Prunkard et al., “High-Level Expression of Recombinant Human Fibrinogen in the Milk of Transgenic Mice,” Nature Biotech. 14:867-871 (1996)). These products are being developed as treatments for arthiritis, HIV/AIDS, cancer, and autoimmune diseases. One limitation of the transgenic process is the long lag-time between cloning and production (˜18 months). However, recent developments have purportedly cut this time in half.

The complexity of milk combined with the low concentration of target protein complicates the recovery process from transgenic milk. Whole milk consists of more than 100,000 different molecules dispersed in three phases namely, lipid, casein, and whey (Tetra Pak Processing Systems, AB, S-221 86, Dairy Processing Handbook, Lund Sweden:Verlag. 452 p. (1995)) and heterologous recombinant proteins can be overproduced in the range of 0.2 to 1 wt. %. Traditional methods used by the dairy industry to isolate proteins from milk involving pasteurization followed by enzymatic coagulation or acid precipitation at pH 4.6 (pI of casein) are unsuitable for the recovery of heterologous proteins because they can be temperature and pH sensitive. Additionally, the coagulation process can trap a large amount of the target protein within the casein pellets resulting in poor yields (Morcol et al., “Model Process for Removal of Caseins from Milk of Transgenic Animals,” Biotechnol. Prog. 17:577-582 (2001)). Transgenic milk is neither pasteurized nor homogenized in order to prevent damage and loss of the target heterologous proteins. In non-homogenized transgenic milk, the liquid fat droplets, ranging from 0.1 to 20 μm in diameter (Goff et al., “Dairy Chemistry and Physics,” In: Hui Y H, editor, Dairy Science and Technology Handbook, Vol. 1, Principles and Properties. New York:VCH. p 1-81 (1993)), are encased by a 8 to 10 nm thick membrane called the native fat globule membrane (FGM). The FGM is composed of phospholipids and proteins and is characterized by a very low interfacial surface tension, 1 to 2.5 mN/m, between the fat globules and the serum phase. This prevents the globules from flocculation and from enzymatic degradation. Homogenization breaks up the fat globules causing disruption of the native FGM, which allows serum proteins and casein micelles to freely adsorb onto the exposed fat globules. This results in loss of heterologous proteins through adsorption (Meade et al., “Recombinant Protein Expression in Transgenic Mice,” In: Femadez J, Hoeffler J, editors, Gene Expression Systems: Using Nature for the Art of Expression, Carlsblad:Academic Press. p 399-427 (1998)). The latter effect is expected to reduce the yield of target protein and the former effect increases membrane fouling because of a lower value of back-transport due to shear or inertial lift of small fat globules. The casein micelle is a roughly spherical, fairly swollen particle of 0.1 to 0.3 μm diameter with a hairy outer layer (Walstra, “Casein Sub-Micelles: Do They Exist?,” Int. Dairy J. 9:189-192 (1999); McMahon, “Rethinking Casein Micelle Structure Using Electron Microscopy,” J. Dairy Sci. 81:2985-2993 (1998)). The hairy layer is comprised of C-terminal ends of κ-casein. This prevents further aggregation of micelles and flocculation by steric and electrostatic repulsion at pH values higher than 4.6, the pI of casein. Thus, at the physiological pH of milk, 6.4-6.6, the casein micelles predominantly exist as distinct particles of a size range comparable to the mean pore size (0.1 μm) of the poly(ether sulfone) microfiltration membrane used here. This is expected to result in a low shear-induced diffusion coefficient as well as fouling by pore blockage and cake formation, especially at low shear rates. Fat globules and casein micelles are retained in whole milk microfiltration, whereas the product protein permeates through the membrane along with the whey proteins, minerals, and sugars. This is corroborated by polyacrylamide gel electrophoresis studies of permeate samples of milk clarified by microfiltration with a 0.2 μm average pore size ceramic membrane which indicate negligible casein transmission through the membrane.

The present invention is directed to an improved procedure for recovering target molecules from polydisperse liquids, including recovering components from milk.

### SUMMARY OF THE INVENTION

One embodiment of the present invention relates to a method of recoverying a target entity from a polydisperse liquid. This method includes subjecting the polydisperse liquid to a microfiltration process utilizing a microfiltration membrane under conditions effective to permit the target entity to pass through the microfiltration membrane. The microfiltered polydisperse liquid is then subjected to an ultrafiltration process utilizing an ultrafiltration membrane under conditions effective to permit the target entity to be retained on the ultrafiltration membrane. This method is effective to cause the target entity to be recovered from the polydisperse liquid in a yield of greater than 75% and a purity of greater than 80%.

Another embodiment of the present invention is directed to a method of recovering a target entity from a polydisperse liquid by subjecting the polydisperse liquid to a microfiltration process. The microfiltration process utilizes a microfiltration membrane under conditions effective to permit the target entity to pass through the microfiltration membrane as a permeate, where the target entity in the permeate is greater than 90% of the target entity present in the polydisperse liquid and the target entity is present in the permeate in a concentration of 7-10%.

Yet another embodiment of the present invention is directed to a method of recovering a target entity from a polydisperse liquid by subjecting the polydisperse liquid to an ultrafiltration process. The ultrafiltration process utilizes an ultrafiltration membrane under conditions effective to permit the target entity to be retained on the ultrafiltration membrane at a pH which differs from the target entity's pI.

A two step microfiltration and ultrafiltration process has been developed for achieving high yield and purity of proteins from high fouling, polydisperse suspensions of biological origin. One such suspension is transgenic whole milk which includes high fouling fat, somatic cells, casein, etc. apart from the target entity. Recovery is fraught with challenges due to the polydispersity and molecular diversity of whole milk. This two step process results in yields in excess of 75% and purity of 80% of a heterlogous therapeutic protein expressed in transgenic milk.

The first step can involve the use of a short helical hollow fiber microfiltration module (average pore size 0.1 micrometers) with approximately uniform transmembrane pressure along the length, achieved by permeate circulation in a co-axial direction (called coflow) as the retentate flow. This ensures that the transmission of the target protein through the membrane is enhanced due to the dual advantages of low transmembrane pressure leading to a sparse cake, and hence higher transmission, as well as the high shear rates inherent in the helical module due to self-cleaning Dean vortices. Additionally, the use of a short module allows operation at higher shear rates within the design pressure of the membrane module. A crucial aspect of this step is operation at the isoelectric pH of the target protein which minimizes charge exclusion of the target protein from membrane pores and cake interstices. This step results in a clear permeate consisting of over 90% of the target protein along with other milk whey proteins, salts, and sugars. The concentration of IgG is in the range of 7 to 10% of the total proteins in TGM. Microfiltration (MF) in the diafiltration mode increases to around 7 to 20%, depending on the extent of pre-concentration prior to diafiltration.

The second step involves an ultrafiltration (UF) scheme to raise the purity, for example, of IgG from 7% to 80% with a yield of 80%. As a 95% yield of target protein was achieved in the MF stage, this resulted in a two step MF/UF process with an overall yield of 75% and a purity of 80% for the target protein. Tangential flow UF experiments in diafiltration mode were conducted with 100 kD cellulosic membranes to evaluate the optimal pH, ionic strength, and uniform transmembrane pressure (TMP). The TMP was kept uniform by permeate circulation in co-flow mode. The traditional approach of conducting the UF close to the pI of the predominant whey proteins (15-40 kD, pI-5.2) to facilitate their ready passage, whereas the bulkier (155 kD) and charged IgG is retained, could not be applied because of precipitation of residual casein at pH values lower than 8.5. Instead, the packing characteristics of the cake layer on the membrane wall was utilized at a pH of 10.75 and 15 mM NaCl to achieve a selectivity of 18, which is sufficient to meet the stated goals of purity and yield for this difficult separation.

The distinctive features of the MF step are the choice of a novel short helical hollow fiber membrane module in lieu of commercially available linear versions, operating pH at the isoelectric pH of the target protein, operation at nearly uniform low transmembrane pressure less than or equal to 2 psi, low axial velocity of 1 m/s, very low permeation flux of less than 30 lmh, and a rapid acid free cleaning regimen of just 45 minutes to ensure full membrane cleaning for reuse. The distinctive features of the UF step are the optimization of ionic strength, pH, and permeation flux to achieve protein separation at a pH different from the pI of any of the proteins involved by using concentration polarization and the correct amount of charge shielding.

### BRIEF DESCRIPTION OF THE DRAWINGS

FIG. 1A shows the linear and new coiled hollow fiber design. FIG. 1B is a flow sheet of the lab-scale hollow fiber microfiltration system showing two separate flow systems.

FIG. 2 is a schematic drawing of a microfiltration system.

FIG. 3 shows the hydraulic permeability versus run number for the short helical hollow fiber module (H-3a) after several cleaning cycles.

FIG. 4A shows the average permeation flux, while FIG. 4B shows the IgG yield versus pH for the short helical hollow fiber module (H-3a) at a uniform TMP of 2.5 psi, an axial velocity of 1.1 m/sec (Re≅1075), a 1× milk concentration, and the number of diavolumes (N_{d})=4.

FIGS. 5A shows the average permeation flux, while FIG. 5B shows the IgG yield versus uniform TMP for the short helical hollow fiber module at 1.1 m/s axial velocity (Re≅1075), pH=9.0, 1× milk conc., and N_{d}=4.

FIG. 6 is a schematic of three different operating regimes during microfiltration of poly-disperse suspensions. Regime I: pore constriction. Regime II: cake consolidation. Regime III: Pressure independent flux.

FIG. 7A shows the yield, while FIG. 7B shows the duration divided by the milk concentration versus the milk concentration for the short helical (H-3a) and linear (L-3a) hollow fiber module at 1.0 m/s axial velocity (Re≅977), pH=9.0, ≦2 psi uniform TMP, and N_{d}=4 for 1×, 5 for 1.25×, and 6 for higher milk concentrations.

FIG. 8 shows the yield versus axial velocity (Re≅830-1170) for the short helical (H-3a) hollow fiber module at pH=9.0, ≦2 psi uniform TMP, 1× milk concentration, and N_{d}=4.

FIG. 9 is a schematic drawing of an ultrafiltration system.

FIG. 10 is a plot of flux (lmh) versus transmembrane pressure (psi).

FIG. 11 is a plot of selectivity versus NaCl concentration (mM).

FIG. 12 is a plot of selectivity versus flux (lmh).

FIG. 13 is a plot of IgG yield versus flux (lmh).

FIG. 14 is a plot of IgG purification factor versus flux (lmh).

FIG. 15 is a plot of IgG purification factor versus time (hours) and IgG yield versus time (hours).

### DETAILED DESCRIPTION OF THE INVENTION

One embodiment of the present invention relates to a method of recoverying a target entity from a polydisperse liquid. This method includes subjecting the polydisperse liquid to a microfiltration process utilizing a microfiltration membrane under conditions effective to permit the target entity to pass through the microfiltration membrane. The microfiltered polydisperse liquid is then subjected to an ultrafiltration process utilizing an ultrafiltration membrane under conditions effective to permit the target entity to be retained on the ultrafiltration membrane. This method is effective to cause the target entity to be recovered from the polydisperse liquid in a yield of greater than 75% and a purity of greater than 80%.

Most existing theories of MF and UF deal with monodisperse suspensions and the pressure independent regime (Belfort et al., “The Behavior of Suspensions and Macromolecular Solutions in Crossflow Microfiltration,” J. Membr. Sci. 96:1-58 (1994) and Zeman et al., Microfiltration and Ultrafiltration Principles and Applications. Marcel Dekker, Inc., New York, (1996), which are hereby incorporated by reference in their entirety), where the dominant resistance is provided by the cake on the membrane wall. Both solvent and solute transport through the membrane are governed by the balance between convection of solutes to the membrane and the back transport of solutes from the membrane wall to the bulk solution by Brownian diffusion, shear induced diffusion or inertial lift mechanisms, and the sieving through the membrane wall. These theories do not predict solute transport, ignore solute-solute and solute-wall interactions, and are valid only for the laminar flow regimes.

Applicants have recently developed a theory called the Aggregate transport model (see PCT Application Seri. No. PCT/US03/25230, filed Aug. 13, 2003, which is hereby incorporated by reference in its entirety), that addresses two crucial aspects missing in the earlier theories: (a) a priori prediction of solute transport and (b) solute polydispersity, which is the rule rather than the exception in real suspensions. This approach builds upon the earlier back-transport models, by incorporating an iterative technique which relates the nature of the cake on the membrane wall to the propensity of back transport of various solutes. Packing constraints, based on geometry, are utilized to determine the nature of the cake. The lowest propensity to back-transport gives the first approximation for the permeation flux. The interstices of the cake are likened to membrane pores. The solute partitioning coefficient, φ, is evaluated as a function of the ratio of the transmitting solute to the diameter of the cake interstice based on hard sphere interactions. The hindrance factor, K_{c}, is evaluated based on simplified versions (Zeman et al., “Polymer Solute Rejection by Ultrafiltration Membranes,” Synthetic Membranes vol. II. Hyperfiltration and Ultrafiltration Uses (A. F. Turbak, ed.), ACS Symposium Series No. 54, American Chemical Society, Washington, D.C., p. 412 (1981), which is hereby incorporated by reference in its entirety). Thus,
φ=(1−λ)² (1)
K_{c}=[2−(1−λ)²]exp(−0.7146λ²) (2)
For high Peclet numbers, the solute flux is thus evaluated by using the convection equation,
Nₛ=φK_{c}VC_{w} (3)
For operation in the pressure dependent regime, the membrane determines both solvent and solute transport. For the idealized geometry of a spherical solute and cylindrical pore, the general expression for the solute partitioning coefficient is (Zydney et al., “Protein Transport Through Porous Membranes: Effect of Colloidal Interactions,” Coll. Surf A. 138:133-143 (1998), which is hereby incorporated by reference in its entirety), φ=2/rₚ².

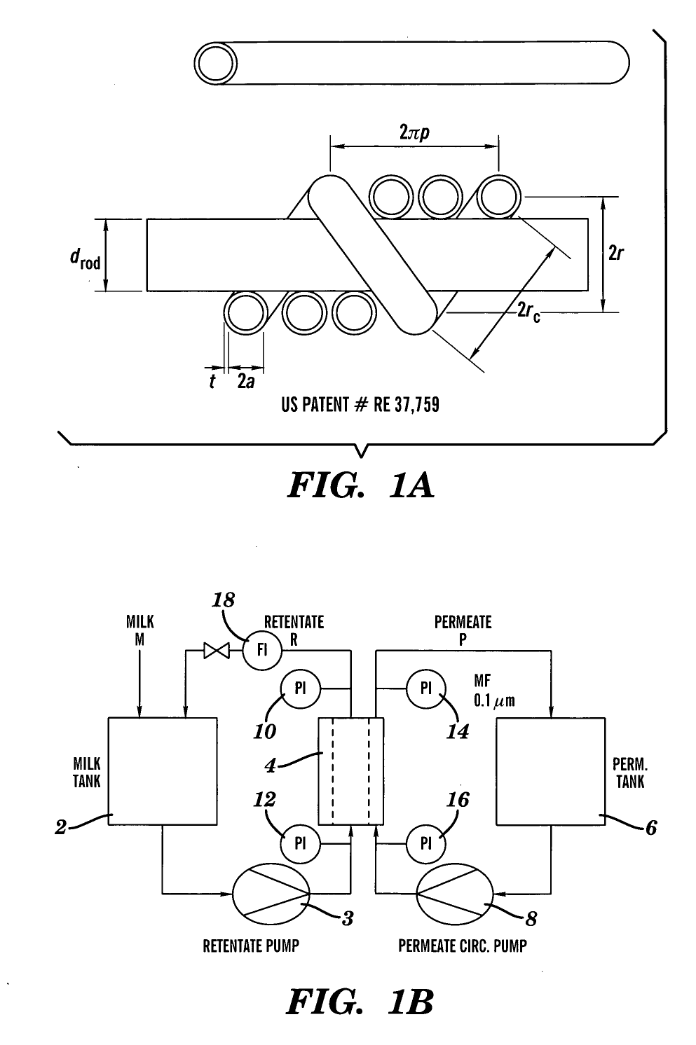

The microfiltration process is preferably carried out using flow around a curved microporous walled membrane channel. Typically, this is achieved with a Dean vortex in a helical hollow fiber membrane module, as shown in FIG. 1A. This procedure is fully described in U.S. Pat. Nos. 5,626,758 and Re 37,759 to Belfort, et. al., which are hereby incorporated by reference in their entirety. The Dean vortex is of sufficient strength to disturb build-up of solute and particles near a surface of the membrane.

The microfiltration process is carried out using co-flow of permeate and retentate. As shown in FIG. 1B, milk M enters milk tank 2 from which it is discharged by retentate pump 3 into microfiltration unit 4. Retentate R is returned from microfiltration unit 4 into milk tank 2 after passing through flow indicator 18. Liquid from permeate tank 6 is withdrawn by permeate circulation pump 8 and passed into microfiltration unit 4. Permeate P is then returned to permeate tank 6. The pressure drop of retentate across microfiltration unit 4 is measured by pressure indicators 10 and 12, while the pressure drop of permeate across microfiltration unit 4 is measured by pressure indicators 14 and 16.

The microfiltration process is carried out at the target entity's isoelectric pH, a transmembrane pressure difference of less than 2 psi, an axial flow rate of less than 1 meter/second, and a permeation flux of less than 30 lmh.

After subjecting the polydisperse liquid to a microfiltration process, the microfiltration membrane is subjected to an acid-free cleaning regime. This is carried out by rinsing with deionized water at an axial flow velocity of 2 m/s for 5 minutes with the permeate ports fully opened. This is followed by recycling cleaning agents Ultrasil 10—detergent at 0.5 wt. % and Ultrasil 02 surfactant at 0.1 wt. % at an axial velocity of 2 m/s at 45° C. for 30 minutes. The cleaning agents (Ultrasil 02, 10, Ecolab, St. Paul, Minn.) are then flushed from the system for 10 minutes with deionized water. This is followed by sterilization with 0.1 wt. % NaOCl at 40° C. for 10 minutes at 0.33 m/sec. This velocity was chosen to give sufficient residence time for the bleach to act on the membrane modules. The membranes are stored in this dilute bleach solution until the next time the microfiltration process is to be carried out, before which the dilute bleach solution is flushed out by rinsing with deionized water for 10 minutes at 2 m/s velocity.

The ultrafiltration process can be carried out by utilizing an ultrafiltration membrane under conditions effective to permit the target entity to be retained on the ultrafiltration membrane at a pH which differs from the target entity's pI. Typically, this involves carrying out the ultrafiltration process at a pH above that at which the target entity precipitates. Particularly suitable ultrafiltration process conditions are at a pH greater than 8.5, preferably greater than 10.

The ultrafiltration process can be carried out at an ionic strength of 10-20 mM NaCl, preferably 12-17 mM NaCl.

The ultrafiltration process can be carried out at a permeation flux of 110-130 lmh, preferably 115-125 lmh.

The target entity is selected from the group consisting of a protein, polypeptide, amino acid, colloid, mycoplasm, endotoxin, virus, carbohydrate, RNA, DNA, and antibody.

Where the target entity is a is protein or polypeptide, it can be selected from the group consisting of glycoprotein, immunoglobulin, hormone, enzyme, serum protein, milk protein, cellular protein, and soluble receptor. Particularly suitable proteins or polypeptides are selected from the group consisting of alpha-proteinase inhibitor, alkaline phosphatase, angiogenin, antithrombin III, chitinase, extracellular superoxide dismutase, Factor VIII, Factor IX, Factor X, fibrinogen, glucocerebrosidase, glutamate decarboxylase, human serum albumin, insulin, myelin basic protein, lactoferrin, lactoglobulin, lysozyme, lactalbumin, proinsulin, soluble CD4, component and complexe of soluble CD4, and tissue plaminogen activator.

The polydisperse liquid is milk produced by a transgenic animal. The transgenic animal is selected from the group consisting of a cow, goat, pig, rabbit, mouse, rat, and sheep.

Procedures for making transgenic animals are well known.

One means available for producing a transgenic animal (e.g., a mouse) is as follows: female mice are mated, and the resulting fertilized eggs are dissected out of their oviducts. The eggs are stored in an appropriate medium such as M2 medium (Hogan B. et al. Manipulating the Mouse Embryo, A Laboratory Manual, Cold Spring Harbor Laboratory (1986), which is hereby incorporated by reference). A DNA or cDNA molecule is purified from a vector (such as plasmids pCEXV-alpha, pCEXV-alpha, or pCEXV-alpha) by methods well know in the art. Inducible promoters may be fused with the coding region of the DNA to provide an experimental means to regulate expression of the transgene. Alternatively or in addition, tissue specific regulatory elements may be fused with the coding region to permit tissue-specific expression of the transgene. The DNA, in an appropriately buffered solution, is put into a microinjection needle (which may be made from capillary tubing using a pipet puller), and the egg to be injected is put in a depression slide. The needle is inserted into the pronucleus of the egg, and the DNA solution is injected. The injected egg is then transferred into the oviduct of a pseudopregnant mouse (i.e., a mouse stimulated by the appropriate hormones to maintain pregnancy but which is not actually pregnant), where it proceeds to the uterus, implants, and develops to term. Alternatively, transgenic animals can be prepared by inserting a DNA molecule into a blastocyst of an embryo or into embryonic stem cells.

The polydisperse liquid can also be a cell culture fluid from transgenic plant cells. The transgenic plant cells are from plants, such as alfalfa, canola, rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery carrot, squash, pumpkin, zucchini, cucumber, apple, pear, melon, strawberry, grape, raspberry, pineapple, soybean, tobacco, tomato, sorghum, sugarcane, or banana.

Procedures for making transgenic plants are well known. In general, methods of making recombinant plant cell(s) involve the introduction of recombinant molecules (e.g., heterologous or not normally present foreign DNA construct) into host cells (e.g., host cells of plant(s), plant tissues, etc.) via specific types of transformation. Thus, a DNA construct contains necessary elements for the transcription and translation in plant cells of an heterologous DNA molecule. The DNA molecule, the promoter, and a 3′ regulatory region can be ligated together using well known molecular cloning techniques as described in Sambrook et al., Molecular Cloning: A Laboratory Manual, Second Edition, Cold Spring Harbor Press, NY (1989), which is hereby incorporated by reference in its entirety.

The DNA construct can be incorporated into cells using conventional recombinant DNA technology. Generally, this involves inserting the DNA construct into an expression vector or system to which it is heterologous (i.e., not normally present). Once the DNA construct of the present invention has been prepared, it is ready to be incorporated into a host cell (e.g., bacteria, virus, yeast, mammalian cells, insect, plant, and the like).

One approach to transforming plant cells and/or plant cell cultures, tissues, suspensions, etc. with a DNA construct of the present invention is particle bombardment (also known as biolistic transformation) of the host cell. This technique is disclosed in U.S. Pat. Nos. 4,945,050, 5,036,006, and 5,100,792, all to Sanford, et al., which are hereby incorporated by reference in its entirety.

Another method of introducing the gene construct of the present invention into a host cell is fusion of protoplasts with other entities, either minicells, cells, lysosomes, or other fusible lipid-surfaced bodies that contain the chimeric gene (Fraley, et al., Proc. Natl. Acad. Sci. USA, 79:1859-63 (1982), which is hereby incorporated by reference in its entirety).

The DNA construct of the present invention may also be introduced into the plant cells and/or plant cell cultures, tissues, suspensions, etc. by electroporation (Fromm, et al., Proc. Natl. Acad. Sci. USA, 82:5824 (1985), which is hereby incorporated by reference in its entirety).

Another method of introducing the DNA construct into plant cells and/or plant cell cultures, tissues, suspensions, etc. is to infect a plant cell with Agrobacterium tumefaciens or Agrobacterium rhizogenes previously transformed with the DNA construct.

Once a recombinant plant cell and/or plant cell cultures, tissues, suspensions, etc. are obtained, it is possible to regenerate a full-grown plant therefrom. Plant regeneration from cultured protoplasts is described in Evans et al., Handbook of Plant Cell Cultures, Vol. 1: (MacMillan Publishing Co., New York, 1983); and Vasil I. R. (ed.), Cell Culture and Somatic Cell Genetics of Plants, Acad. Press, Orlando, Vol. I, 1984, and Vol. III (1986), which are hereby incorporated by reference in their entirety.

Another embodiment of the present invention is directed to a method of recoverying a target entity from a polydisperse liquid by subjecting the polydisperse liquid to a microfiltration process. The microfiltration process utilizes a microfiltration membrane under conditions effective to permit the target entity to pass through the microfiltration membrane as a permeate, where the target entity in the permeate is greater than 90% of the target entity present in the polydisperse liquid and the target entity is present in the permeate in a concentration of 7-10%. The operating conditions of the microfiltration process and the target entity treated in that process are described above.

Yet another embodiment of the present invention is directed to a method of recoverying a target entity from a polydisperse liquid by subjecting the polydisperse liquid to an ultrafiltration processs. The ultrafiltration process utilizes an ultrafiltration membrane under conditions effective to permit the target entity to be retained on the ultrafiltration membrane at a pH which differs from the target entity's pI. The operating conditions of the ultrafiltration process and the target entity treated in that process are described above.

### EXAMPLES

### Example 1

### Feed Suspension

Transgenic goat milk was supplied by GTC Biotherapeutics, from their goat farm in Central, Mass. The average composition of the transgenic goat milk is shown Table 1.

TABLE 1
Composition and Properties of Transgenic Goat Milk

Composition (wt. %)
Fat: 3.5%; Proteins: 3.1% (80% casein, rest α-lactalbumin,
β-lactoglobulin, Immunoglobulins); Lactose: 4.6%;
Ash: 0.8%; Human IgG: 2 to 3 g/l; Total solids: 12%.
Salient properties
pH: 6.6-6.9; Target human IgG pI: 9.0; Casein pI: 4.6
Main component diameters
Fat: 1 to 20 μm; Casein micelles: 0.3-0.4 μm; IgG: 20 nm (155 kDa);
Other whey proteins: 15-40 kDa

The human IgG concentration in the transgenic goat milk (˜8 g/l) was diluted with non-transgenic milk to between 1.75 to 3.25 g/l.

### Example 2

### Membrane Modules

Tubular hollow fiber membrane were supplied by Millipore Corporation, Bedford, Mass. Each module had six 0.1 μm mean pore-size polyether sulfone hollow fibers of internal diameter 1.27 mm and pore diameter of 100 mn. For the helical module, the fibers were wound in a single-wrap helix around an acrylic rod (FIG. 1A). The lengths of the linear (L-3a) and helical (H-3a) modules were 18.5 and 13.5 cm, respectively. The corresponding filtration areas were 44 and 32 cm².

### Example 3

### Flowsheet

The flow diagram of the labscale hollow fiber microfiltration system is given in FIG. 1B. This system consists of two circulation loops—retentate and permeate. The retentate loop consists of the milk tank (any graduated tube of 50 ml capacity), a hollow fiber microfiltration module as described in Example 1, a peristaltic pump (Masterflex, 7521, Cole Parmer, Chicago, Ill.), two pressure gauges (glycerine filled, 0-60 psi, 1008, Aschcroft, Stratford, Conn.) upstream and downstream of the hollow fiber microfiltration module, a flowmeter (tube size #14, Gilmont, Barrington, Ill.), and a needle valve downstream of the flowmeter. The axial velocity of the retentate stream and the back pressure downstream of the microfiltration module can be varied in this loop. The permeate circulation loop consists of the permeate tank (any glass graduated flask of 500 ml capacity), a peristaltic pump (Masterflex, 7521, Cole Parmer, Chicago, Ill.), and two pressure gauges (glycerine filled, 0-60 psi, 1008, Aschcroft, Stratford, Conn.) upstream and downstream of the hollow fiber microfiltration module. By adjusting the rpm of the peristaltic pump in the permeate circulation loop, the pressure drop in the tube and shell side of the hollow fiber microfiltration module can be made nearly equal by coflow permeate circulation in the shell side of the microfiltration module. This facilitates a low uniform TMP along the microfiltration module. Without the permeate circulation loop, the TMP and the axial velocity cannot be independently varied. A high axial velocity would lead to a high pressure upstream of the microfiltration module leading to a high non-uniform TMP along the microfiltration module. The hold up volume in the retentate loop is 85 ml, whereas in the permeate loop it was between 25 and 50 ml depending on the microfiltration module used.

### Example 4

### Diafiltration Experiments

A series of diafiltration experiments ( 4 to 6 diavolumes) with pH adjusted deionized water were conducted to study the IgG transmission and permeation flux behavior with the passage of diafiltration and with respect to variation in operating pH, TMP, concentrating factor, module type, and axial velocity. For the pH experiments, the pH of milk was adjusted to the values 7.5. 8, 8.5, and 9 by adding requisite quantities of 0.25 M NaOH solution according to the milk pH calibration graph prepared earlier (pH=2.857[NaOH](g/l)+6.7436, R²=0.99). The base values of the other variables were 2.5 psi uniform TMP, axial velocity of 1.1 m/sec, 1× milk concentration, and short helical module. To study the effect of TMP, the TMP was varied from 2 psi to 4.5 psi at the optimum pH determined in the previous step. Milk at concentrations corresponding to 1, 1.25, 1.5, 1.75, and 2 times the normal milk concentration were used to investigate the effect of concentrating factor on protein transmission. For 1× concentration, diafiltration was started after flushing the system with milk. For 2× concentration, one system volume was collected prior to diafiltration, while maintaining a constant reservoir level with milk addition. The permeate volumes collected in the concentration phase for 1.25×, 1.5×, 1.75×, and 2× were 0.25, 0.5, 0.75, and 1 times the retentate loop volume, respectively. To compare the efficacy of the linear module with the helical module, experiments were conducted in the diafiltration mode at five concentrations corresponding to 1, 1.25, 1.5, 1.75, and 2 times the normal milk concentration with the short linear module. Samples (1 ml) were taken from the retentate and permeate streams at regular intervals and analyzed for IgG and protein concentrations. Finally, the effect of axial velocity on the target protein transmission was studied using a short helical module at 1× milk concentration, 2 psi uniform TMP, pH=9, and velocities ranging from 0.85 to 1.2 m/sec (Re≅830−1170, Re =2au_{ave.}/v, where 2a (m) is the internal diameter of the membrane bore, uₐᵥₑ (m/s) is the average axial velocity in the membrane bore and v(m²/s) is the kinematic viscosity of milk). All experiments were conducted in the laminar axial flow regime.

### Example 5

### IgG Assay and Yield

This assay is based on the protocol supplied by GTC Biotherapeutics, (Framingham, Mass.) which is described as follows. Protein A affinity chromatography (PA ImmunoDetection™ sensor cartridge (2.1×30 mm), PerSeptive Biosystems, Framingham, Mass.) was used to obtain IgG concentrations in the various goat milk streams. 1.5 ml of milk samples were pipetted into 2 ml Eppendorf centrifuge tubes and centrifuged at 21000 g for 30 min. The milk separated into a top fat layer, a clear whey solution, and a casein pellet. 0.75 ml of the clear whey phase was carefully extracted with a pipette after puncturing the fat layer. This was pipetted into centrifuge tubes (catalog # 8163, Spin-X tubes, Corning, N.Y.) with 0.45 μm pore size cellulose acetate membranes and were centrifuged at 2100 g for 15 min. The clarified permeate was then injected into the HPLC column. For the permeate samples, sample preparation was unnecessary. A HPLC (Waters 510 with Millennium 2010 operating system) with a 486 UV detector and U6K sample injector were used (Waters Corp., Milford, Mass.). The loading buffer was 10 mM phosphate buffer, 150 mM NaCl at pH 7.20±0.05 and the elution buffer was 12 mM HCl with 150 mM NaCl. The pump flow rate was set at 2 ml/min. and the detector wave length at 280 nm. The injection volume was 10 μL for milk and 20 to 40 μL for permeate samples. A calibration graph was constructed by injecting different dilutions of IgG fusion protein (GTC Biotherapeutics, Framingham, Mass.). Loading buffer was passed through the column for 10 minutes followed by sample injection and loading buffer again for 5 minutes. After this, elution buffer was run for 10 minutes. A clean peak corresponding to IgG fusion protein was detected at around 6.5 minutes into the elution phase. Area obtained by peak integration was compared with the calibration graph to obtain the IgG concentration of the sample after dividing by the sample volume. Care was taken to ensure that all readings were within the range of the calibration graph. This was done by adjusting the sample injection quantities. This system has an efficiency of 95% as reported by GTC. There was also some variability in the IgG concentration obtained from milk samples due to small errors in pipetting out the clear solution after centrifuging. The yields for these experiments were computed based on applying a factor of 1.05 to the highest reading of 3.1 g/l obtained from a milk sample with the IgG product. This gives a starting IgG concentration of 3.25 g/l for milk. The yield for a diafiltration experiment was calculated with the formula
Y=(N_{d}<Cp>)/3.25X (2),
where N_{d} is the number of diavolumes, <Cp> is the average permeate IgG concentration, and X is the concentration factor of milk prior to diafiltration.

### Example 6

### Protein Assay

The Bradford assay (# 500-0006, Bio-Rad, Hercules, Calif.) was used to determine protein concentration. Bovine lyophilized casein powder (C7891, Sigma, St. Louis, Mo.) was used as a standard and readings were taken in disposable 5 ml polystyrene cuvettes (# 223-9950, Bio-Rad). The absorbance readings with the spectrophotometer (U-2000, Hitachi, Japan) were taken in the visible range at 595 nm wavelength.

### Example 7

### Fat Assay

Fat content was measured by the Gerber method which is approved for use by dairies in USA. 11 ml of preheated milk sample (37° C.) was added to 10 ml of sulfuric acid in a butyrometer. 1 ml of amyl alcohol was added and the butyrometer was capped with a special stopper. Shaking the butyrometer ensures digestion of the proteins by sulfuric acid. The butyrometer was then inverted and centrifuged for 6 minutes at 350 g. After this, the butyrometer was immersed in a water bath at 65° C. for 5 min. The fat appeared as a clear liquid and the quantity was read out as a volume percentage in the graduated section of the butyrometer.

### Example 8

### Cleaning Regimen

The following cleaning protocol was used. After each experiment, the coflow pump and tubing were detached and the system was rinsed with deionized water at 50° C. at an axial flow velocity of 1.3 m/s for 1 minute with the permeate ports fully opened. This was followed by recycling cleaning agents Ultrasil 10—detergent at 0.5 wt. % and Ultrasil 02—surfactant at 0.1 wt. % at an axial velocity of 1.3 m/s at 50° C. for 30 minutes. The loss in volume due to permeation was made up by addition of deionized water at 55° C. The cleaned membrane deionized water fluxes are plotted in FIG. 3. This indicates very good flux recovery from run to run.

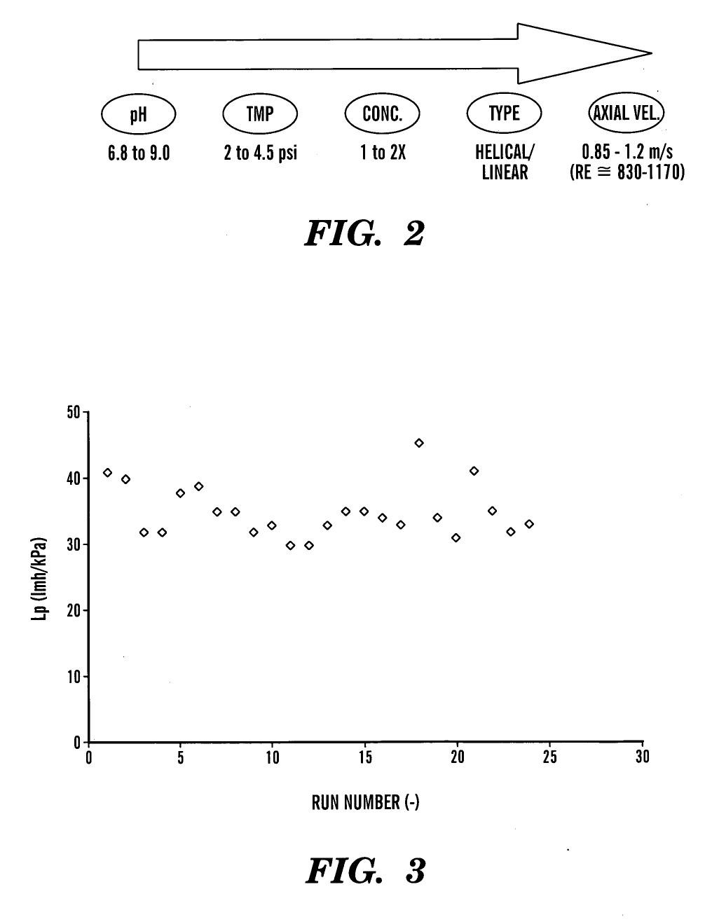

A series of experiments to evaluate base values of variables was first run. Then, the optimization strategy described in FIG. 2 was followed to successively optimize the yield of the target protein, one variable at a time.

### Example 9

### Initial Experiments

Several experiments were conducted to evaluate the yield of the target protein (IgG) with various TMP's, linear, helical and ceramic microfiltration modules, with and without coflow and at different pH values. These results are shown in Table 2.

TABLE 2
Initial Yields of IGG

| Product | Module^{a} | Conc.^{b} | Mode^{c} | pH^{d} | Yield(%)^{e} |
|---|---|---|---|---|---|
| IgG | Linear | 1X | Standard | 6.8 | 0.7 |
| IgG | Helical | 1X | Coflow | 6.8 | 3 |
| IgG | Ceramic | 1X | Coflow | 6.8 | 3.6 |
| IgG | Helical | 1X | Standard | 9 | 9 |
| IgG | Ceramic | 1X | Coflow | 9 | 36 |
| ^{a}Linear: Traditional linear hollow fiber module; Helical: Specially designed hollow fiber module where the fibers are helically wound around a support rod. Relatively short linear (L-3a) and helical (H-3a) modules were chosen. | | | | | |
| ^{b}Concentration: Factor by which the milk is concentrated in the experiment. 1X means the normal concentration of milk. | | | | | |
| ^{c}Standard: Normal mode of crossflow membrane filtration where the feed is circulated axially in the bore of the hollow fibers and the permeate at atmospheric pressure is drawn from the shell side of the hollow fiber module; Coflow: special mode of crossflow membrane filtration where the permeate is circulated in the shell side in the same direction as the retentate, resulting in similar pressure drops in the bore of the tube and shell side leading to approximately | | | | | |
| # uniform transmembrane pressure in the axial direction of the hollow fiber membrane module. | | | | | |

^{d}pH of the milk feed and diafiltration buffer.
^{e}Yield: Ratio of the mass of product harvested in the permeate by the mass of product fed into the system.

The yield values were very low for the first four cases with a large improvement for the experiment conducted at pH=9.0, the isoelectric point of the target IgG as confirmed by GTC Biotherapeutics. This demonstrated that the operating pH was indeed a very important variable.

### Example 10

### Microfiltration Optimization Experiments

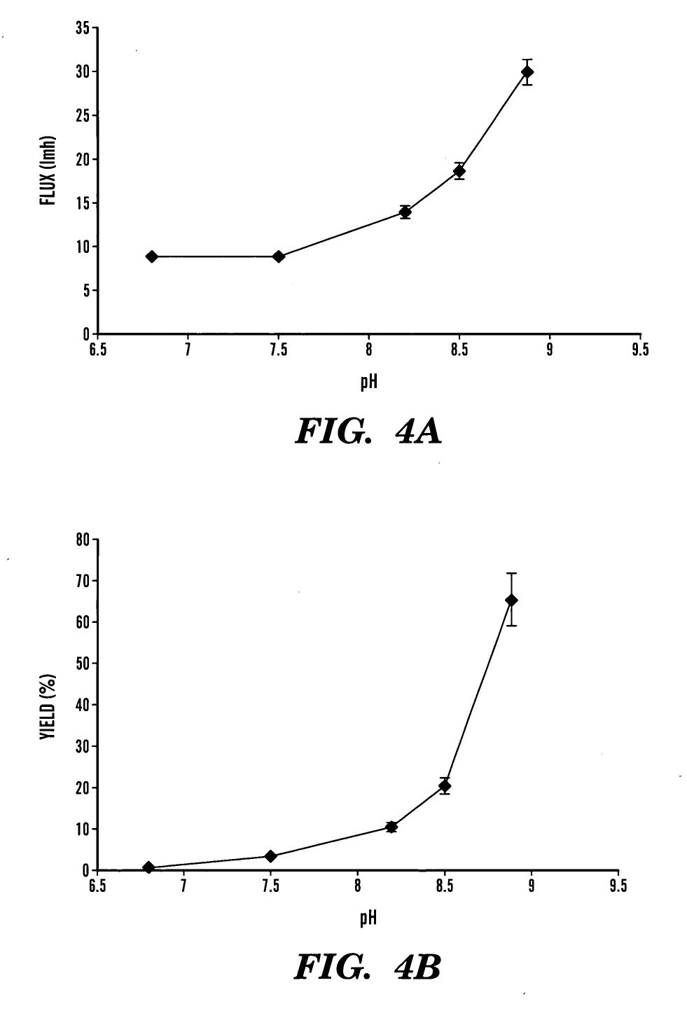

A series of pH experiments were conducted with the short helical module (H-3a, area=32 cm²) with co-flow at a uniform TMP of 2.5 psi, 1× milk concentration, and axial velocity of 1.1 m/sec (Re≅1075). The average permeation flux increased more than threefold from below 10 to over 30 lmh as the pH was raised from the physiological value of 6.8 to 9.0, isoelectric point of the target IgG (FIG. 4A). IgG yield increased dramatically from 0.7% to around 70% with increase in pH (FIG. 4B). This confirmed applicants' hypothesis of high transmission of target protein at its pI and is consistent with the results reported by other researchers (Bums et al., “Effect of Solution pH on Protein Transport through Ultrafiltration Membranes,” Biotech. & Bioengg. 64:27-37 (1999) and Bums et al., “Contributions to Electrostatic Interactions on Protein Transport in Membrane Systems,” AIChE J. 47:1101-14 (2001), which are hereby incorporated by reference in their entirety). This is due to a low value of electrical potential of the target molecules leading to lower exclusion from membrane pores and cake interstices. It was decided to set the operating pH at 9.0. Higher values of pH were not considered as it was deemed better to retain an operating pH as close to the physiological pH of milk which is 6.8.

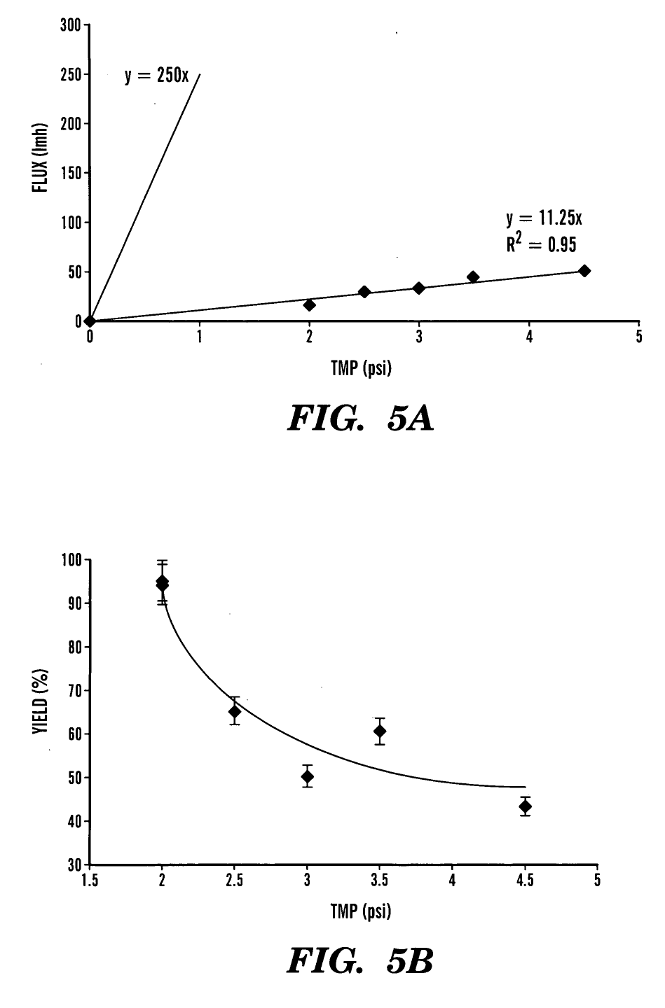

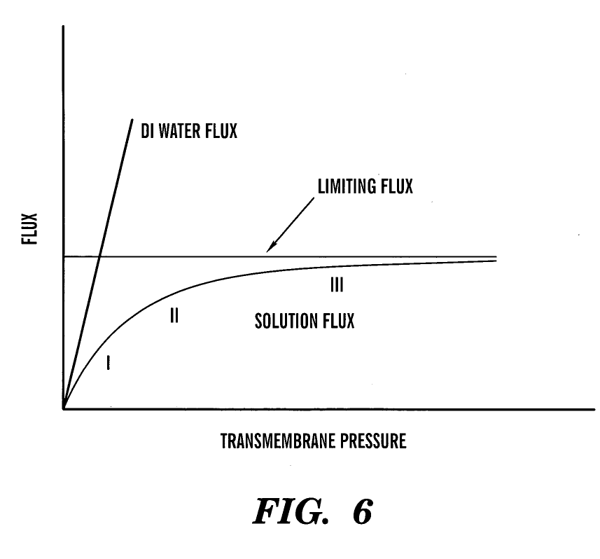

However, to further increase the product yield, experiments were conducted to improve the mass transfer characteristics of the system. Several experiments were conducted with the short helical module (H-3a, area =32 cm²) at pH 9.0, 1× milk concentration, axial velocity of 1.1 m/sec (Re ≅1075), and uniform TMP's in the range of 2-4.5 psi (FIG. 5). The average permeation flux varied linearly with TMP. This confirmed applicants' selection of the operating regime as the pressure-dependent regime, where the permeation flux varies linearly with TMP (FIG. 6). In this regime, the cake deposit on the membrane was expected to be sparse thereby facilitating good protein transmission. This hypothesis was confirmed by the experimental results. At 2 psi, 95% yields were achieved, in duplicate, whereas at the higher TMP of 4.5 psi the yield dropped to just 40%. The permeation flux, on the other hand rose with increasing TMP, from 16 to 50 lmh. As the product, in this case, is costly, a very high yield with a low permeation flux is considered more desirable than a low yield with a high permeation flux. Hence, the optimum uniform TMP is selected as 2 psi giving an average permeation flux of 16 lmh and 95% yield after four diavolumes. For other products, an optimization exercise could be carried out to obtain the desired combination of high flux with reasonable product yields.

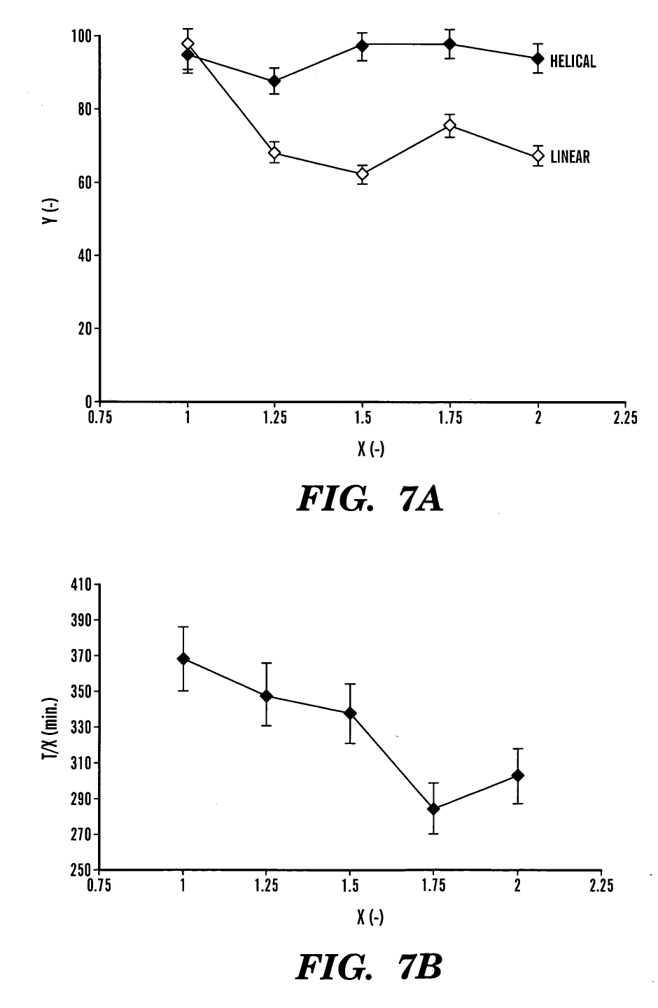

A series of diafiltration experiments with different feed milk concentration factors of 1× to 2× were then conducted with a short helical module (H-3a, area=32 cm² ) at pH 9.0, uniform TMP of 2 psi, and axial velocity of 1 m/sec. The average yield for these experiments was over 95% (FIG. 7A). Four diavolumes were sufficient for the 1× experiment whereas 5 to 6 diavolumes were necessary for the experiments with higher concentration. Concentration was achieved by incorporating a concentration step prior to diafiltration with deionized water at pH 9.0. For instance, 2× concentration was achieved by adding milk instead of deionized water for the first diavolume to the reservoir. At higher milk concentrating factors, the average permeation flux decreases and the number of diavolumes to achieve an IgG yield of 95% goes up. However, this is balanced out by the fact that at larger milk concentrations, a higher mass of product protein is recovered. Thus, there is a need to optimize the milk concentrating factor, to obtain the condition where the product recovery per unit time is maximized. To obtain the optimum diafiltration conditions Ng et al., “Optimization of Solute Separation by Diafiltration,” Sep. Sci. II(5):499-502 (1976), which is hereby incorporated by reference in its entirety), a plot was constructed between diafiltration time/milk concentration factor, T/X, and milk concentration factor, X (FIG. 7B). This accounts for the higher quantities of milk processed at higher concentrations. The minimum of this plot gives the best operating point, that is, ˜1.75×.

To compare the efficiency of the linear module with the helical module, similar experiments were conducted in the diafiltration mode at various milk concentrating factors, with the short linear module with all the other variables at the previously determined optimal values (FIG. 7A). Except for 1× concentration, where the yield was in excess of 95%, for all the other concentrations the yields were moderate (60 to 75%). This clearly indicates that the linear module was not as effective as the helical module for handling concentrated suspensions. This was possibly due to the greater concentration of fat globules at the membrane wall for the linear modules which operate at a lower wall shear rate at the same axial velocity as the helical modules.

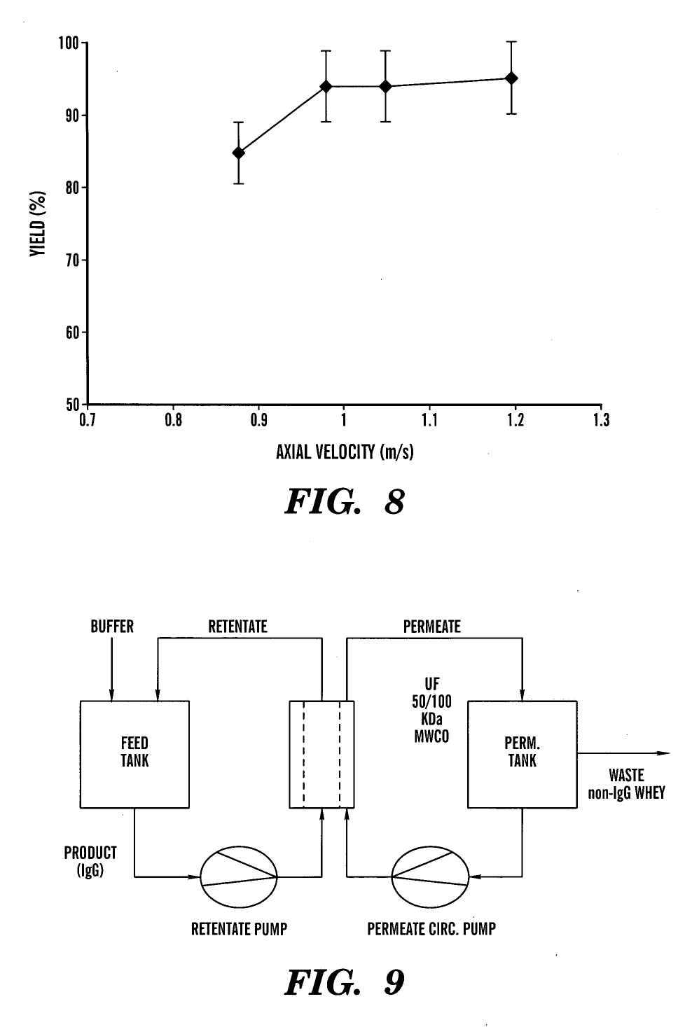

Finally, the effect of axial velocity was evaluated with the short helical module (H-3a; area=32 cm²) at pH 9.0, uniform TMP of ≦2 psi, 1× milk concentration, and various axial velocities in the range 0.85 to 1.2 m/sec corresponding to Reynold's numbers 830 to 1170 (FIG. 8). 95% yield was obtained for velocities ≧0.95 m/sec. The best operating velocity was 0.95 m/sec (Re≅930).

Thus the optimum conditions determined were pH 9.0, uniform TMP of 2 psi, 1.75× milk concentration factor, helical module, and 0.95 m/sec. axial velocity resulting in 95% yield of the target IgG.

The methodology reported here demonstrates that by combining optimum fluid mechanics with optimum electrostatics, the yield of a target protein from a highly complex polydisperse suspension like whole transgenic goat milk could be raised from an extremely low initial value of 0.7% to a value of 95%. The following are recommended:
1. Use a short path length microfiltration module.
2. Operate at a low uniform TMP.
3. Operate with the helical hollow fiber microfiltration module at the combination of all the optimizing conditions reported here to obtain low membrane fouling and high yield of the target protein.
4. Start-up so that the permeation flux is in the region of 10 lmh (for the helical module) and does not rise above this value even transiently, by adjusting the TMP. The permeation flux rises as diafiltration proceeds, to give an average value of around 15 lmh due to decrease in feed viscosity and solids concentration.

The results presented here demonstrate that high product recoveries are possible in microfiltration of complex poly-disperse suspensions by careful control of the various parameters and selection of the appropriate module geometry as detailed in the methodology presented here. The helical microfiltration module appears to be superior to the commercially available linear version when handling concentrated suspensions. The results and the methodology described here should be generalizeable to other complex suspensions of biological origin.

### Example 11

### Ultrafiltration Optimization Experiments

The scheme employed for the ultrafiltration (UF) step is shown in FIG. 9. The permeate obtained after microfiltration of transgenic milk contains the desired product at a purity (mass ratio of product to total protein) of about 7%. The UF step is proposed to raise the purity to 80% with a yield of 80% to give an overall process yield of 75% and a purity of 80% for the combined MF/UF process. A selectivity (defined as the ratio of the observed sieving coefficients of the other whey proteins and the target IgG) of 18 and a purification factor (defined as P =(Final concentration of IgG in the retentate/Initial concentration of IgG in the retentate)/(Final concentration of non-IgG proteins in the retentate/Initial concentration of non-IgG proteins in the retentate) of 50 to achieve this. This is based on mass balance of solutes (van Reis et al., “Optimization Diagram for Membrane Separations,” J. Membr. Sci. 129:19-29 (1997), which is hereby incorporated by reference in its entirety). The target IgG (155 kDa molecular weight) has a pI of 9 whereas the predominant whey proteins have pI's in the range of 4.5 to 5.2 (MW 14 to 36 kDa). The usual practice would be to adjust the feed pI to around 5 so that IgG gets retained in the 100 kDa cut off UF membrane whereas the other proteins readily permeate through based both on size and charge.

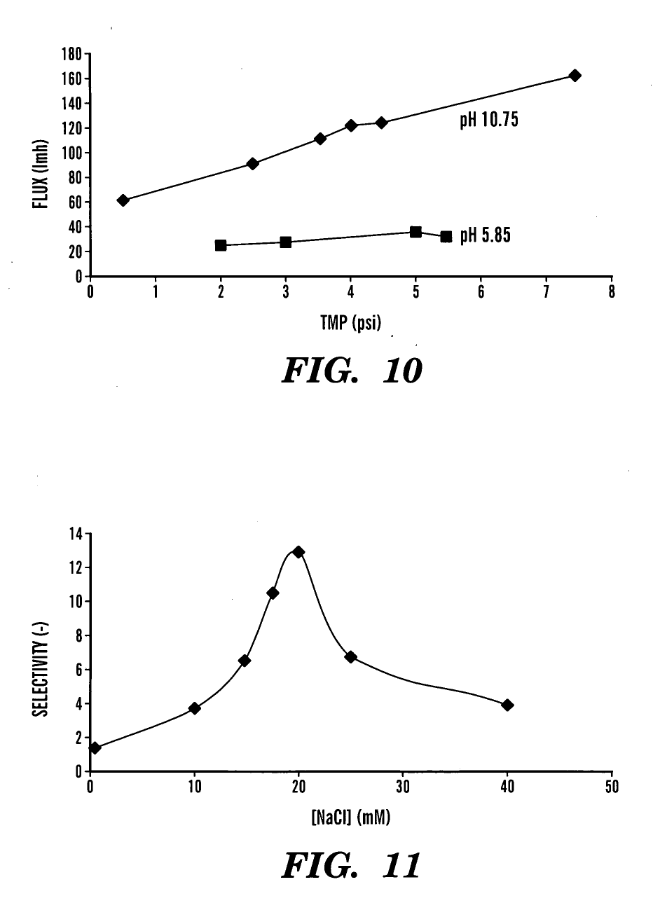

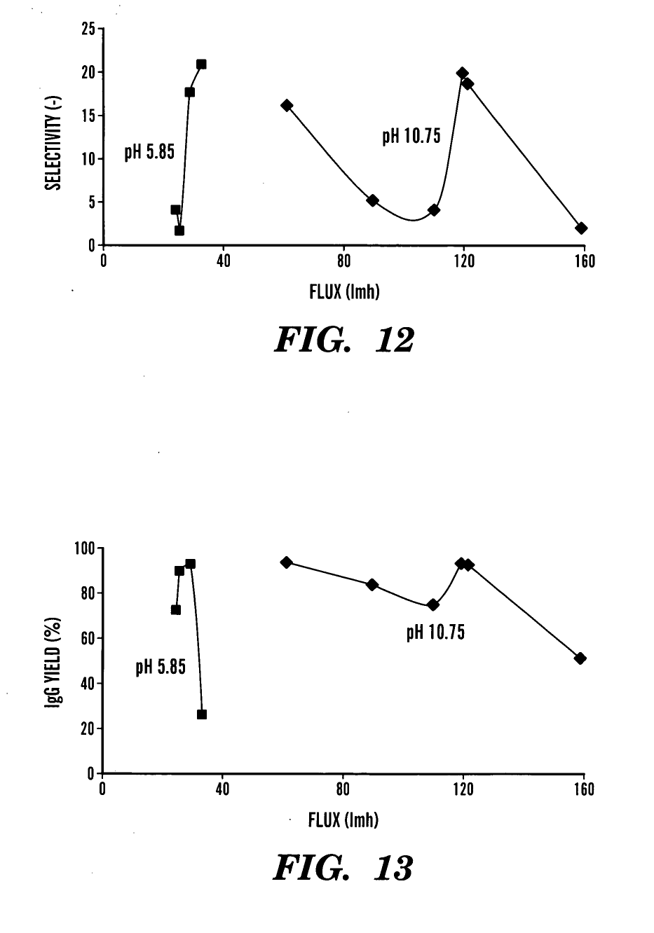

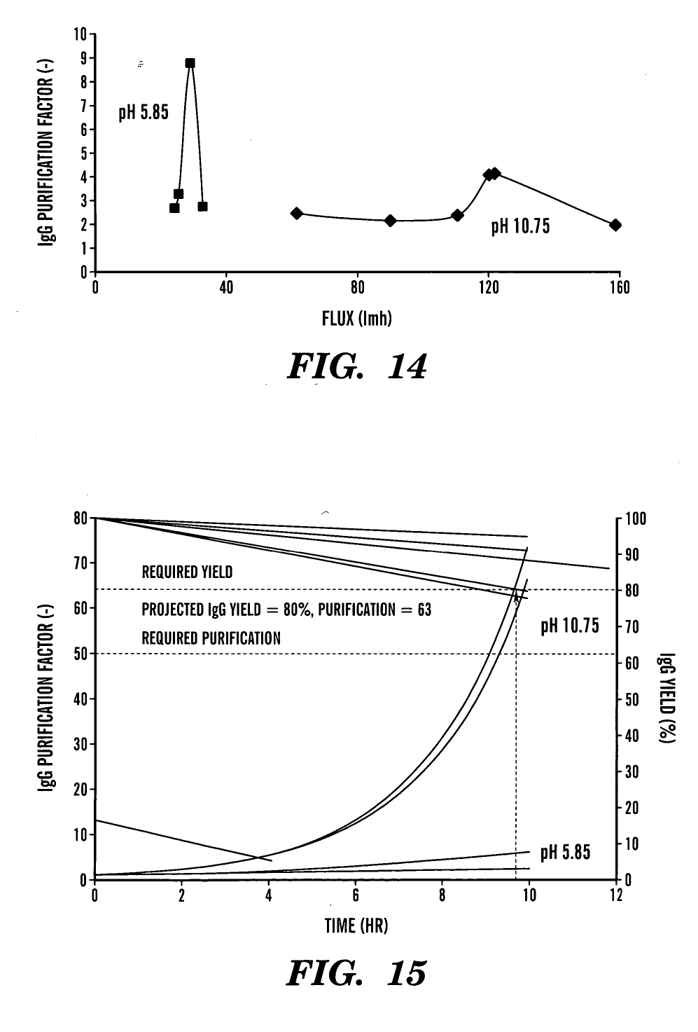

This cannot be readily applied here, because of precipitation at pH below 8.5. To increase protein solubility (salting in) 100 mM NaCl was added. Thus, it was possible to do the UF at pH ranging from 5.2 to 11. It was found that high selectivities were achieved at pH values of 10.75 and 5.85. A pH of 10.75 was chosen as a much higher flux could be obtained in comparison with pH 5.85 (FIG. 10). Apart from pH, the other two optimizing variables were ionic strength and permeation flux. At any pH, high ionic strength leads to increase charge shielding and thinner double layers around the protein molecules. At pH 11, the optimum ionic strength was 20 mM NaCl (FIG. 11). The corresponding value at pH 10.75 was 15 mM NaCl. A higher ionic strength leads to greater passage of IgG leading to lower selectivity and loss of product whereas a lower ionic strength leads to lower passage of IgG but the other whey proteins as well which are far away from their pI's of around 4.5 to 5.2. Thus, an ionic strength of less than 15 mM leads to low selectivity between IgG and other whey proteins as both species are retained to a large degree. The effect of permeation flux is subtle. A low flux will lead to a sparse cake as elucidated in the aggregate transport model (Baruah et al., “A Predictive Aggregate Transport Model for Microfiltration of Combined Macromolecular Solutions and Poly-Disperse Suspensions: Model Development,” Biotechnol. Progress, 19:1524-32 (2003) and Baruah et al., “A Predictive Aggregate Transport Model for Microfiltration of Combined Macromolecular Solutions and Poly-Disperse Suspensions: Testing Model with Transgenic Goat Milk,” Biotechnol. Progress, 19:1533-40 (2003), which are hereby incorporated by reference in their entirety) on the membrane and lead to higher permeation of the bigger IgG molecules leading to a low selectivity and low yield. Again, at very high permeation rates, IgG leaks out through the UF membrane because of the higher IgG concentration at the wall, high pressure differential, and high concentration difference across the membrane. Hence, an intermediate permeation flux (120 lmh) leads to low IgG transport coupled with a moderate transport of other whey proteins leading to the favorable combination of high selectivity, yield and purity (FIGS. 12, 13, and 14). The projected yield and purification factor of IgG based on extrapolation in time are shown to exceed the required values of 80% each for pH 10.75. (FIG. 15).

Although the invention has been described in detail for the purpose of illustration, it is understood that such details are solely for that purpose and variations can be made therein by those skilled in the art without departing from the spirit and scope of the invention which is defined by the following claims.

1. A method of recoverying a target entity from a polydisperse liquid, said method comprising:
subjecting the polydisperse liquid to a microfiltration process utilizing a microfiltration membrane under conditions effective to permit the target entity to pass through the microfiltration membrane and
subjecting the microfiltered polydisperse liquid to an ultrafiltration process utilizing an ultrafiltration membrane under conditions effective to permit the target entity to be retained on the ultrafiltration membrane, whereby the target entity is recovered from the polydisperse liquid in a yield of greater than 75% and a purity of greater than 80%.

2. The method of claim 1, wherein the microfiltration process is carried out using flow around a curved microporous walled membrane channel.

3. The method of claim 3, wherein the microfiltration process is carried out in a helical hollow fiber membrane module which produces Dean vortices of sufficient strength to disturb build-up of solute and particles near a surface of the membrane.

4. The method of claim 1, wherein the microfiltration process is carried out using co-flow of permeate and retentate.

5. The method of claim 1, wherein the microfiltration process is carried out at the target entity's isoelectric pH.

6. The method of claim 1, wherein the microfiltration process is carried out at a transmembrane pressure difference of less than 2 psi.

7. The method of claim 1, wherein the microfiltration process is carried out at an axial flow rate of less than 1 meter/second.

8. The method of claim 1, wherein the microfiltration process is carried out at a permeation flux of less than 30 lmh.

9. The method of claim 1, wherein the microfiltration process is carried out using a Dean vortex in a helical hollow fiber membrane module, co-flow of permeate and retentate, the target entity's isoelectric pH, a transmembrane pressure difference of less than 2 psi, an axial flow rate of less than 1 meter/second, and a permeation flux of less than 30 lmh.

10. The process of claim 1, wherein the microfiltration process is carried out using the target entity's isoelectric pH, a transmembrane pressure difference of less than 2 psi, an axial flow rate of less than 1 meter/second, and a permeation flux of less than 30 lmh.

11. The method of claim 1, wherein the target entity is selected from the group consisting of a protein, polypeptide, amino acid, colloid, mycoplasm, endotoxin, virus, carbohydrate, RNA, DNA, and antibody.

12. The method of claim 11, wherein the target entity is an antibody.

13. The method of claim 11, wherein the target entity is protein or polypeptide.

14. The method of claim 13, wherein protein or polypeptide is selected from the group consisting of glycoprotein, immunoglobulin, hormone, enzyme, serum protein, milk protein, cellular protein, and soluble receptor.

15. The method of claim 13, wherein protein or polypeptide is selected from the group consisting of alpha-proteinase inhibitor, alkaline phosphatase, angiogenin, antithrombin III, chitinase, extracellular superoxide dismutase, Factor VIII, Factor IX, Factor X, fibrinogen, glucocerebrosidase, glutamate decarboxylase, human serum albumin, insulin, myelin basic protein, lactoferrin, lactoglobulin, lysozyme, lactalbumin, proinsulin, soluble CD4, components or complexes of soluble CD4, and tissue plaminogen activator.

16. The method of claim 1, wherein the polydisperse liquid is milk produced by a transgenic animal.

17. The method of claim 16, wherein the transgenic animal is selected from the group consisting of a cow, goat, pig, rabbit, mouse, rat, and sheep.

18. The method of claim 1, wherein the polydisperse liquid is cell culture fluid from transgenic plant cells.

19. The method of claim 18, wherein the transgenic plant cells are from plants selected from the group consisting of alfalfa, canola, rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery carrot, squash, pumpkin, zucchini, cucumber, apple, pear, melon, strawberry, grape, raspberry, pineapple, soybean, tobacco, tomato, sorghum, sugarcane, and banana.

20. The method of claim 1 further comprising:
subjecting the microfiltration membrane to an acid-free cleaning regime after said subjecting the polydisperse liquid to a microfiltration process.

21. The method of claim 1, wherein the ultrafiltration process is carried out by utilizing an ultrafiltration membrane under conditions effective to permit the target entity to be retained on the ultrafiltration membrane at a pH which differs from the target entity's pI.

22. The method of claim 1, wherein the ultrafiltration process is carried out at a pH above that at which the target entity precipitates.

23. The method of claim 22, wherein the ultrafiltration process is carried out at a pH greater than 8.5.

24. The method of claim 22, wherein the ultrafiltration process is carried out at a pH greater than 10.

25. The method of claim 1, wherein the ultrafiltration process is carried out at an ionic strength of 10-20 mM NaCl.

26. The method of claim 25, wherein the ultrafiltration process is carried out at an ionic strength of 12-17 mM NaCl.

27. The method of claim 1, wherein the ultrafiltration process is carried out at a permeation flux of 100-130 lmh.

28. A method of recoverying a target entity from a polydisperse liquid, said method comprising:
subjecting the polydisperse liquid to a microfiltration process utilizing a microfiltration membrane under conditions effective to permit the target entity to pass through the microfiltration membrane as a permeate, whereby the target entity in the permeate is greater than 90% of the target entity present in the polydisperse liquid and the target entity is present in the permeate in a concentration of 7-20%.

29. The method of claim 28, wherein the microfiltration process is carried out using flow around a carved microporous walled channel membrane.

30. The method of claim 29, wherein the microfiltration process is carried out in a helical hollow fiber membrane module which produces Dean vortices of sufficient strength to disturb build-up of solute and particles near a surface of the membrane.

31. The method of claim 28, wherein the microfiltration process is carried out using co-flow of permeate and retentate.

32. The method of claim 28, wherein the microfiltration process is carried out at the target entity's isoelectric pH.

33. The method of claim 28, wherein the microfiltration process is carried out at a transmembrane pressure difference of less than 2 psi.

34. The method of claim 28, wherein the microfiltration process is carried out at an axial flow rate of less than 1 meter/second.

35. The method of claim 28, wherein the microfiltration process is carried out at a permeation flux of less than 30 lmh.

36. The method of claim 28, wherein the microfiltration process is carried out using a Dean vortex in a helical hollow fiber membrane module, co-flow of permeate and retentate, the target entity's isoelectric pH, a transmembrane pressure difference of less than 2 psi, an axial flow rate of less than 1 meter/second, and a permeation flux of less than 30 lmh.

37. The process of claim 28, wherein the microfiltration process is carried out using the target entity's isoelectric pH, a transmembrane pressure difference of less than 2 psi, an axial flow rate of less than 1 meter/second, and a permeation flux of less than 30 lmh.

38. The method of claim 28, wherein the target entity is selected from the group consisting of a protein, polypeptide, amino acid, colloid, mycoplasm, endotoxin, virus, carbohydrate, RNA, DNA, and antibody.

39. The method of claim 38, wherein the target entity is an antibody.

40. The method of claim 38, wherein the target entity is protein or polypeptide.

41. The method of claim 40, wherein protein or polypeptide is selected from the group consisting of glycoprotein, immunoglobulin, hormone, enzyme, serum protein, milk protein, cellular protein, and soluble receptor.

42. The method of claim 40, wherein protein or polypeptide is selected from the group consisting of alpha-proteinase inhibitor, alkaline phosphatase, angiogenin, antithrombin III, chitinase, extracellular superoxide dismutase, Factor VIII, Factor IX, Factor X, fibrinogen, glucocerebrosidase, glutamate decarboxylase, human serum albumin, insulin, myelin basic protein, lactoferrin, lactoglobulin, lysozyme, lactalbumin, proinsulin, soluble CD4, component and complex of soluble CD4, and tissue plaminogen activator.

43. The method of claim 28, wherein the polydisperse liquid is milk produced by a transgenic animal.

44. The method of claim 43, wherein the transgenic animal is selected from the group consisting of a cow, goat, pig, rabbit, mouse, rat, and sheep.

45. The method of claim 28, wherein the polydisperse liquid is cell culture fluid from transgenic plant cells.

46. The method of claim 45, wherein the transgenic plant cells are from plants selected from the group consisting of alfalfa, canola, rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery carrot, squash, pumpkin, zucchini, cucumber, apple, pear, melon, strawberry, grape, raspberry, pineapple, soybean, tobacco, tomato, sorghum, sugarcane, and banana.

47. The method of claim 28 further comprising:
subjecting the microfiltration membrane to an acid-free cleaning regime after said subjecting the polydisperse liquid to a microfiltration process.

48. A method of recoverying a target entity from a polydisperse liquid, said method comprising:
subjecting the polydisperse liquid to an ultrafiltration processs utilizing an ultrafiltration membrane under conditions effective to permit the target entity to be retained on the ultrafiltration membrane at a pH which differs from the target entity's pI.

49. The method of claim 48, wherein the ultrafiltration process is carried out at a pH above that at which the target entity precipitates.

50. The method of claim 49, wherein the ultrafiltration process is carried out at a pH greater than 8.5.

51. The method of claim 50, wherein the ultrafiltration process is carried out at a pH greater than 10.

52. The method of claim 48, wherein the ultrafiltration process is carried out at an ionic strength of 10-20 mM NaCl.

53. The method of claim 48, wherein the ultrafiltration process is carried out at a permeation flux of 100-130 lmh.

54. The method of claim 48, wherein the target entity is selected from the group consisting of a protein, polypeptide, amino acid, colloid, mycoplasm, endotoxin, virus, carbohydrate, RNA, DNA, and antibody.

55. The method of claim 54, wherein the target entity is an antibody.

56. The method of claim 54, wherein the target entity is protein or polypeptide.

57. The method of claim 56, wherein protein or polypeptide is selected from the group consisting of glycoprotein, immunoglobulin, hormone, enzyme, serum protein, milk protein, cellular protein, and soluble receptor.

58. The method of claim 56, wherein protein or polypeptide is selected from the group consisting of alpha-proteinase inhibitor, alkaline phosphatase, angiogenin, antithrombin III, chitinase, extracellular superoxide dismutase, Factor VIII, Factor IX, Factor X, fibrinogen, glucocerebrosidase, glutamate decarboxylase, human serum albumin, insulin, myelin basic protein, lactoferrin, lactoglobulin, lysozyme, lactalbumin, proinsulin, soluble CD4, component and complex of soluble CD4, and tissue plaminogen activator.

59. The method of claim 48, wherein the polydisperse liquid is milk produced by a transgenic animal.

60. The method of claim 59, wherein the transgenic animal is selected from the group consisting of a cow, goat, pig, rabbit, mouse, rat, and sheep.

61. The method of claim 48, wherein the polydisperse liquid is cell culture fluid from transgenic plant cells.

62. The method of claim 61, wherein the transgenic plant cells are from plants selected from the group consisting of alfalfa, canola, rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery carrot, squash, pumpkin, zucchini, cucumber, apple, pear, melon, strawberry, grape, raspberry, pineapple, soybean, tobacco, tomato, sorghum, sugarcane, and banana.
